(19) United States
(12) Patent Application Publication
(10) Pub. No.: US 2025/0248753 A1
(43) Pub. Date: Aug. 7, 2025

(54) MEDICAL DEVICES AND RELATED METHODS FOR CRYOABLATION

(71) Applicant: BOSTON SCIENTIFIC MEDICAL DEVICE LIMITED, Galway (IE)

(72) Inventors: Nachiket GOLE, Pune (IN); Samriddhi JAIN, Delhi (IN); Arun ADHIKARATH BALAN, Aluva (IN); Subodh MOREY, Ponda (IN); Shrikant Vasant RAUT, Pune (IN); Nidhi DHINGRA, Gurgaon (IN)

(73) Assignee: BOSTON SCIENTIFIC MEDICAL DEVICE LIMITED, Galway (IE)

(21) Appl. No.: 19/046,987

(22) Filed: Feb. 6, 2025

Related U.S. Application Data
(60) Provisional application No. 63/550,878, filed on Feb. 7, 2024.

(51) Int. Cl. A61B 18/02; A61B 18/00

(52) U.S. Cl. CPC A61B 18/02; A61B 2018/0022; A61B 2018/00511; A61B 2018/00517; A61B 2018/00577; A61B 2018/00744; A61B 2018/0212; A61B 2018/0293

(57) ABSTRACT

Medical devices and related methods useful for cryoablation are described. The medical device may include a shaft defining a lumen and having a distal end portion that includes an inflatable balloon; and a tube movable along the lumen. The tube may be in fluid communication with the balloon and configured to supply a cryogenic fluid to a needle closing a distal end of the shaft and to the balloon. The medical device may be movable between a first configuration in which a distal end of the tube abuts a proximal end of the needle while the balloon is deflated, and a second configuration in which the distal end of the tube is proximal to the balloon, wherein supplying the cryogenic fluid through the tube inflates the balloon in the second configuration.

### CROSS-REFERENCE TO RELATED APPLICATIONS

This application claims the benefit of priority to U.S. Provisional Application No. 63/550,878, filed on Feb. 7, 2024, which is incorporated by reference herein in its entirety.

### TECHNICAL FIELD

Aspects of the present disclosure generally relate to medical devices, systems, and methods related thereto for medical procedures. In particular, some aspects relate to medical systems, devices, and methods for cryoablation to treat tissues.

### BACKGROUND

Tissue cancers such as bladder and kidney cancer can be difficult to treat due to the delicate nature of the tissue and constricted anatomy of the urinary system. Urothelial carcinoma is cancer that starts in the urothelium, the tissue that lines parts the urinary system. Urothelial carcinoma accounts for about 90% of all cases of bladder cancer and 7% of all kidney cancer including cancer in renal pelvis and ureter. Treatment usually involves surgery or laser ablation, both of which can include risks and complications for the patient. For example, the ureter is a thin tube that typically cannot handle heat very well due to the risk of necrosis of healthy tissues.

### SUMMARY

The present disclosure includes medical devices and methods useful for cryoablation. The medical device may include a shaft defining a lumen and having a distal end portion that includes an inflatable balloon, wherein a needle closes a distal end of the shaft. The device may also include a tube movable along the lumen. The tube may be in fluid communication with the balloon and may be configured to supply a cryogenic fluid to the needle and to the balloon. The medical device may be movable between a first configuration in which a distal end of the tube abuts a proximal end of the needle while the balloon is deflated, and a second configuration in which the distal end of the tube is proximal to the balloon. Supplying the cryogenic fluid through the tube may inflate the balloon in the second configuration.

According to some aspects, the medical device may include at least one port, e.g., a wall of the distal end portion of the shaft may include the at least one port, that provides fluid communication between the lumen and the balloon. In some examples, the at least one port includes two ports. For example, the two ports may be disposed opposite one another. The balloon, when inflated, may be symmetric or asymmetric about the shaft. The balloon may comprise polyimide, polyamide, polyurethane, nylon, or polyethylene terephthalate. The balloon may have a length ranging from about 8 mm to about 15 mm and/or a diameter ranging from about 8 mm to about 15 mm. In some aspects, the balloon, when deflated, has a plurality of preformed folds, the balloon being configured to inflate with a bellows shape. In some aspects, the balloon, when inflated, has an oblong shape. The medical device may include a handle at a proximal end of the shaft. The handle may include a valve, e.g., a pressure relief valve, configured to control a flow of the cryogenic fluid. The medical device may be used to treat a target tissue site by cryoablation. For example, the cryogenic fluid may comprise nitrogen, nitrous oxide, argon, krypton, or a hydrocarbon refrigerant. In some aspects, the target tissue site is in a bladder or a kidney.

The present disclosure also includes a medical device comprising a shaft defining a lumen and having a distal end portion that includes a port and an inflatable balloon, wherein a needle closes a distal end of the shaft proximate the balloon and the port. The medical device may also include a tube movable along the lumen. The tube may be in fluid communication with the balloon and may be configured to supply a cryogenic fluid to the needle and to the balloon. The medical device may be movable between a first configuration in which a distal end of the tube abuts the needle while the balloon is deflated, and a second configuration in which the distal end of the tube is proximal to the port, wherein supplying the cryogenic fluid through the tube inflates the balloon in the second configuration. According to some aspects, the medical device may include two ports. Additionally or alternatively, the balloon, when deflated, may have a plurality of preformed folds. For example, the plurality of preformed folds may permit the balloon to inflate with a bellows shape.

The present disclosure also includes a method of treating a subject, e.g., using a medical device as described above and/or elsewhere herein. For example, the method may include introducing a medical device into a bodily lumen of the subject, wherein the medical device includes a shaft defining a lumen with a distal end portion that includes an inflatable balloon, wherein a needle closes a distal end of the shaft proximate the balloon. The method may also include at least partially inserting the needle into a target tissue site and supplying a cryogenic fluid to at least one of the needle or the balloon through a tube movable along the lumen of the medical device. The cryogenic fluid may freeze at least a portion of tissue in contact with the needle and/or the balloon. In some aspects, the cryogenic fluid may comprise nitrogen, nitrous oxide, argon, krypton, or a hydrocarbon refrigerant. The distal end of the tube may be adjacent to a proximal end of the needle when supplying the cryogenic fluid. The distal end of the tube may be proximal to the balloon when supplying the cryogenic fluid, such that the balloon inflates. Supplying the cryogenic fluid to the needle and/or the balloon may include supplying the cryogenic fluid through the tube while the balloon is deflated and a distal end of the tube abuts a proximal end of the needle, the distal end of the tube being distal to a port of the shaft in fluid communication with the balloon; moving the tube in a proximal direction such that the distal end of the tube is proximal to the port; and supplying the cryogenic fluid through the tube to inflate the balloon. The bodily lumen may be a urethra and/or the target tissue site may be in a bladder or a kidney.

### BRIEF DESCRIPTION OF THE FIGURES

The accompanying drawings, which are incorporated in and constitute a part of this application, illustrate exemplary aspects that, together with the written descriptions, serve to explain the principles of this disclosure. Each figure depicts one or more exemplary aspects according to this disclosure, as follows:

FIG. 1 depicts an exemplary medical device inserted through the urethra into the bladder, according to some aspects of the present disclosure.

FIGS. 2A-2C depict an exemplary medical device including a needle and a balloon, according to some aspects of the present disclosure.

FIGS. 3A-3D illustrate cross-sectional views of the medical device of FIGS. 2A-2C in different configurations.

FIG. 4 depicts another exemplary medical device and inflation of a balloon, according to some aspects of the present disclosure.

FIG. 5 depicts another exemplary medical device, according to some aspects of the present disclosure.

FIGS. 6A and 6B illustrate use of a medical device according to some aspects of the present disclosure, in a first configuration (FIG. 6A) and a second configuration (FIG. 6B).

### DETAILED DESCRIPTION

Reference will now be made in detail to aspects and examples of the present disclosure and illustrated in the accompanying drawings. Wherever possible, the same reference numbers will be used throughout the drawings to refer to the same or like parts.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

The present disclosure includes medical devices and systems useful for cryoablation by rapid freezing of tissues at and/or proximate a target site. For example, the target site may include tissues that are cancerous or suspected of being cancerous. The medical devices herein may provide an operator (e.g., physician or other medical professional) the ability to treat tissues using the device in a needle configuration, e.g., primarily contacting tissue with a distal needle tip of the device, and/or in a balloon configuration, e.g., primarily or additionally contacting tissue in a wider region by inflation of the balloon proximate the needle. For example, the needle may allow the device to reach target sites deeper into tissues, while the balloon may allow the device to contact a larger area of tissue. While aspects of the present disclosure refer to tissues of the urinary system, such as the bladder and/or kidney, it is understood that the devices and methods herein may be used to treat other types of tissues, including tissues of the gastrointestinal system. Further, while the devices herein may be used to treat cancerous tissue, it is understood that the present disclosure is not limited to cancer treatment.

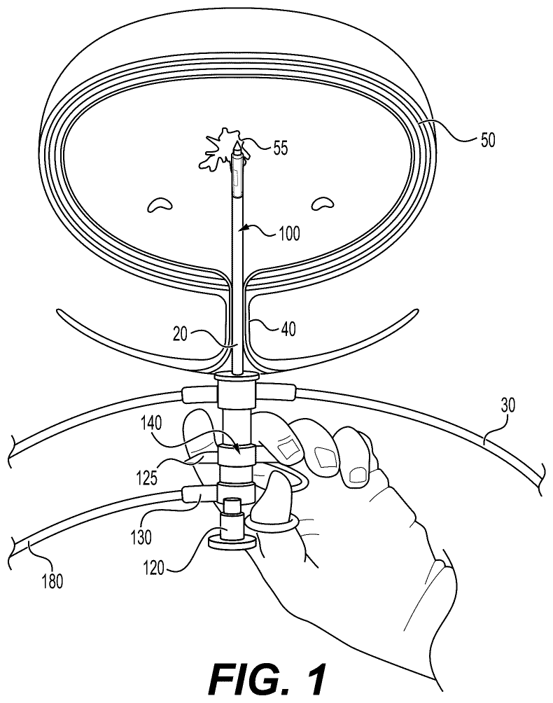

The medical devices herein may be introduced into a bodily lumen through a suitable catheter, e.g., a flexible ureteroscope or endoscope, to reach a target site for treatment. For example, FIG. 1 shows an exemplary medical device 100 introduced through a sheath such as catheter 20 into the bladder 50 of a subject (e.g., patient) to treat a target site 55. Catheter 20 may facilitate insertion of a shaft of medical device 100 into a bodily lumen (e.g., urethra 40 and/or ureter) of the subject, e.g., moving the shaft through the bodily lumen while protecting surrounding tissue. In some examples, catheter 20 may have an outer diameter ranging from about 3 mm to about 6 mm. Catheter 20 may be coupled to a frame 30 configured to couple to an exterior portion of the subject, such as at an insertion site on skin of the subject. The target site 55 may include tissues that are cancerous or suspected of being cancerous. As discussed further below, medical device 100 may be used to deliver cryogenic fluid to the target site 55. The cryogenic fluid may be supplied via tubing 180 coupled to medical device 100, e.g., via a hub or adapter 130 on a handle 120 of medical device 100. The flow fluid may be controlled via an actuator 125 of handle 120. Optionally, actuator 125 may include a valve 140 to control fluid flow.

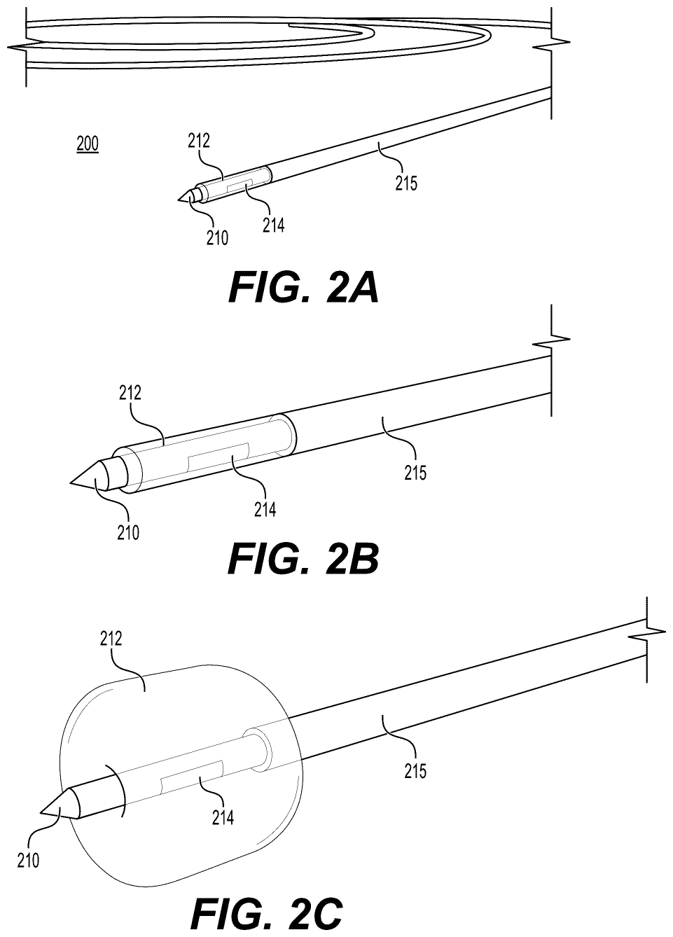

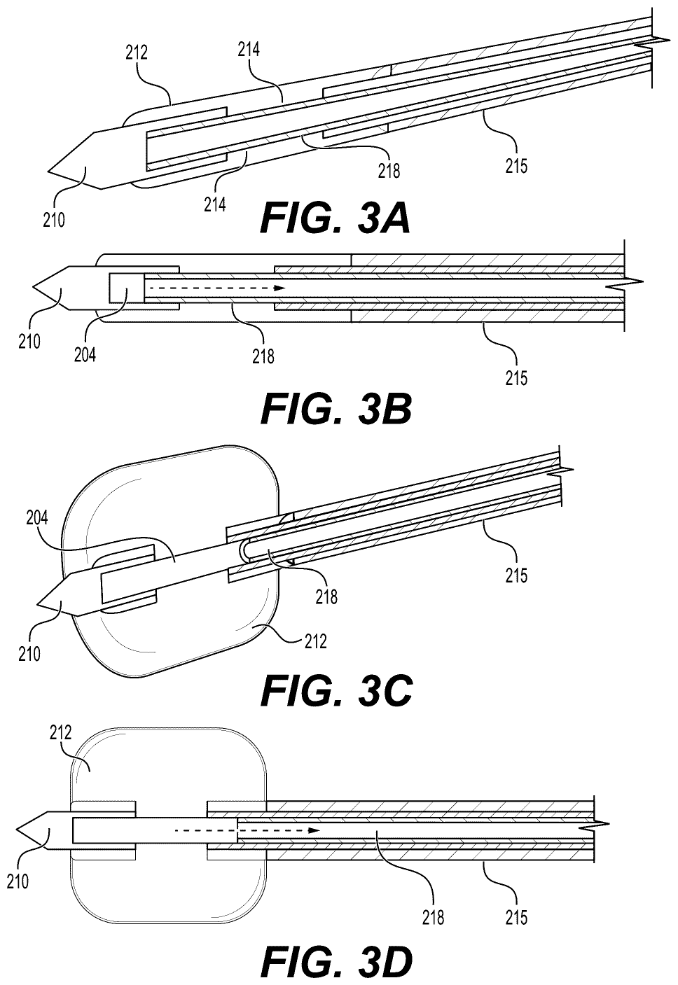

FIGS. 2A-2C illustrate the distal end portion of an exemplary medical device 200 which may include any of the features of medical device 100 (including, e.g., a handle similar to handle 120, etc.). Medical device 200 includes a shaft 215 defining a lumen 204 (see FIGS. 3A-3D), the distal end of shaft 215 and lumen 204 being closed by a needle 210. Needle 210 may have a sharp tip capable of piercing tissue, e.g., to reach tissues below the surface of an organ or other wall of tissue. Exemplary materials suitable for needle 210 include, but are not limited to, biocompatible metals and metal alloys such as stainless steel and Nitinol. Shaft 215 of medical device 200 may comprise a biocompatible material such as a polymer, the shaft having sufficient flexibility to navigate through tortuous anatomy.

Medical device 200 also includes a balloon 212 proximate the distal end, e.g., proximate needle 210, balloon 212 being disposed on or incorporated into the outer surface of shaft 215. Balloon 212 may be inflatable radially outward via a fluid delivered through lumen 204 of shaft 215, e.g., a cryogenic fluid. For example, a distal end portion of shaft 215 proximal to needle 210 may include at least one port 214 in communication with balloon 212. While one port 214 is visible in FIGS. 2A-2C, medical device 200 may include two ports 214 (see FIGS. 3A-3D). The two ports 214 may be disposed opposite one another, e.g., on opposite sides of shaft 215.

Medical device 200 may include a tube 218 (see FIGS. 3A-3D) movable along lumen 204, e.g., to selectively control delivery of the fluid to needle 210 and/or to balloon 212 via port 214. The flow of cryogenic fluid to needle 210 and/or balloon 212 may be controlled by an actuator of medical device 200, e.g., similar to actuator 125 of medical device 100. In some examples, medical device 200 includes a valve, similar to valve 140 of medical device 100. The valve may be a pressure relief valve. Exemplary cryogenic fluids useful for the present disclosure include, but are not limited to, nitrogen, nitrous oxide, argon, krypton, and hydrocarbon refrigerant such as, e.g., octafluoropropane. The cryogenic fluid may be in liquid or gas form.

Balloon 212 may comprise a flexible and/or elastic material or combination of materials that is/are biocompatible and able to withstand low temperatures by the presence of the cryogenic fluid as inflation medium. For example, balloon 212 may comprise an elastic polymer, e.g., a thermoplastic elastomer. The polymer may be natural or synthetic. Exemplary materials suitable for the balloon include, but are not limited to polyimide, polyurethane, nylon elastomers, polyethylene terephthalate, and other thermoplastic elastomers and polymers.

The size of balloon 212 when inflated may be controlled by the amount of cryogenic fluid provided to 212 balloon through lumen 204. In some examples, balloon 212 when fully inflated may have a length along a longitudinal axis of shaft 215 ranging from about 8 mm to about 15 mm, such as about 10 mm to about 12 mm. Additionally or alternatively, balloon 215 when fully inflated may have a width (e.g., a diameter) in a direction perpendicular to the longitudinal axis ranging from about 8 mm to about 15 mm, such as about 10 mm to about 12 mm. Balloon may be configured to inflate symmetrically or asymmetrically about shaft 215 and/or when inflated may have a shape that is symmetric or asymmetric about shaft 215.

As discussed above, the medical devices of the present disclosure are configured to transition between different configurations. FIGS. 3A-3D show cross-sectional views of the distal end portion of medical device 200 wherein tube 218 is retracted in a proximal direction away from needle 210 to move from a first configuration in which the cryogenic fluid is directed to needle 210, and a second configuration in which the cryogenic fluid inflates the balloon 212. In the first configuration illustrated in FIG. 3A, the distal end of tube 218 of abuts a proximal end of needle 210. In this configuration, port 214 providing communication between lumen 204 of shaft 215 to balloon 212 is closed, and balloon 212 is deflated. An operator may retract tube 218 in a proximal direction, e.g., using an actuator of the handle of medical device 200, to pull the distal end of tube 218 proximally away from needle 210, shown in FIG. 3B. Once the distal end of tube 218 reaches port(s) 214 in communication with balloon 212, the cryogenic fluid may flow through port(s) 214 to inflate balloon 212, shown in FIGS. 3C and 3D. In this second configuration, the distal end of tube 218 is proximal to port(s) 214 and may be proximal to balloon 212. In this second configuration, the cryogenic fluid may flow through port(s) 214 to inflate balloon 212.

Once treatment of the tissue is complete, the supply of cryogenic fluid may be terminated and negative pressure applied through lumen 204 to withdraw the fluid and deflate balloon 212. For example, the supply of cryogenic fluid may be replaced with a source of vacuum, or a Y-type connector used to alternate between a supply of cryogenic fluid or a source of vacuum. With balloon 212 deflated, medical device 200 may be removed from the subject, e.g., via the same bodily lumen.

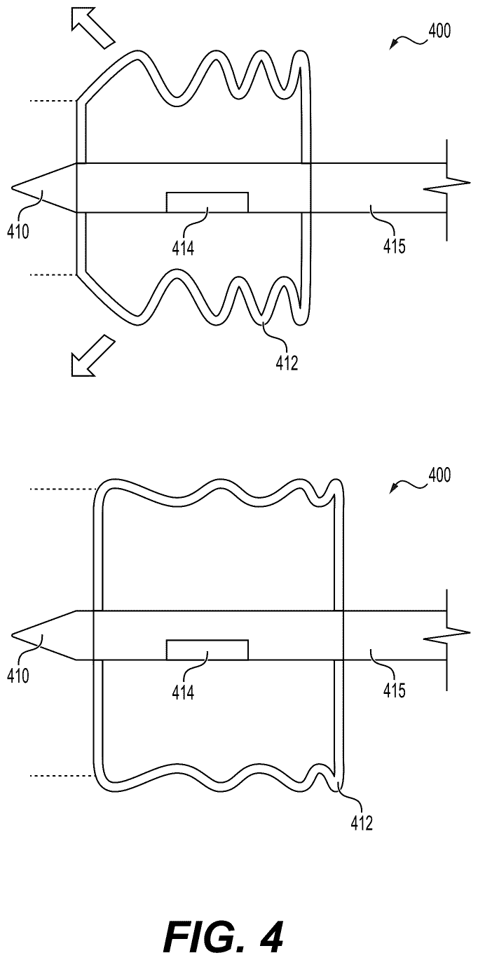

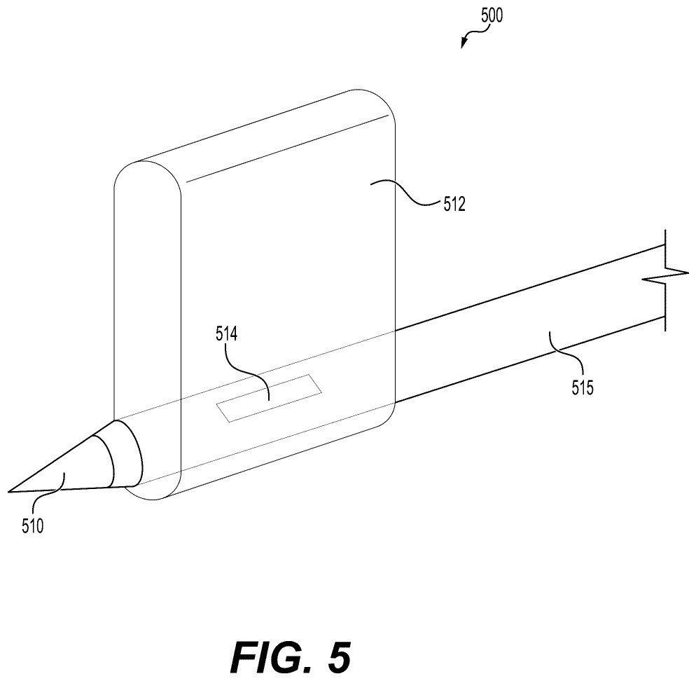

While FIGS. 2A-2C and 3A-3D illustrate a balloon with a generally symmetrical, cylindrical shape, other shapes are also contemplated herein. FIGS. 4 and 5 illustrate other types of balloons that may be used in connection with the present disclosure, including any of the features of medical device 100 and/or 200. For example, the balloon, when inflated, can have different shapes such as, e.g., spherical or oblong shaped. Further, in some examples, the balloon may be configured to inflate such that a portion of the balloon inflates before other portions of the balloon. In this way, the balloon may be configured to contact particular regions or areas of tissue about the shaft of the medical device.

According to some aspects of the present disclosure, the balloon may include one or more preformed folds that determine or influence how the balloon inflates, e.g., the shape of the balloon when partially inflated and/or when fully inflated. For example, the material that forms that balloon may include a plurality of folds such that the balloon inflates in a bellows shape, as illustrated in FIG. 4. For instance, FIG. 4 shows the distal end portion of another exemplary medical device 400 including a shaft 415, a needle 410 and a balloon 412 in communication with a lumen of the shaft 415 via one or more ports 414. Medical device 400 may include any of the features of medical device 100 and/or medical device 200. In this example, balloon 412, when, deflated includes a plurality of folds at or proximate the proximal end of balloon 412. Thus, when cryogenic fluid is provided via port 414, balloon 412 begins to inflate more fully at the distal end of balloon 412 (top image of FIG. 4) than at the proximal end with the folds. As inflation continues, the cryogenic fluid continues to inflate the remainder of balloon 412, e.g., gradually smoothing out the folds at the proximal end (bottom image of FIG. 4).

FIG. 5 shows the distal end portion of another exemplary medical device 500 including a shaft 515, a needle 510 and a balloon 512 in communication with a lumen of the shaft 515 via one or more ports 514. Medical device 500 may include any of the features of medical device 100, medical device 200, and/or medical device 400. In this example, balloon 512 is shown inflated with an asymmetric shape about shaft 515. For example, balloon 512 illustrates an oblong shape.

The size and shape of the balloon may be selected based on the nature of the tissue to be treated, e.g., the size and shape of the target site. For example, a symmetric, cylindrical shape similar to that shown in FIGS. 2A-2C may be useful for treating a relatively large area proximate and surrounding the distal end (e.g., needle) of the shaft of the medical device. In another example, a bellows-type shape (whether symmetric as shown in FIG. 4 or asymmetric) may be useful for primarily targeting tissue proximate the needle of the shaft with less contact of tissue surrounding the proximal end of the balloon. Further, for example, an asymmetric shape such as that shown in FIG. 5 may be useful to primarily target tissue on one side of the distal end of the shaft.

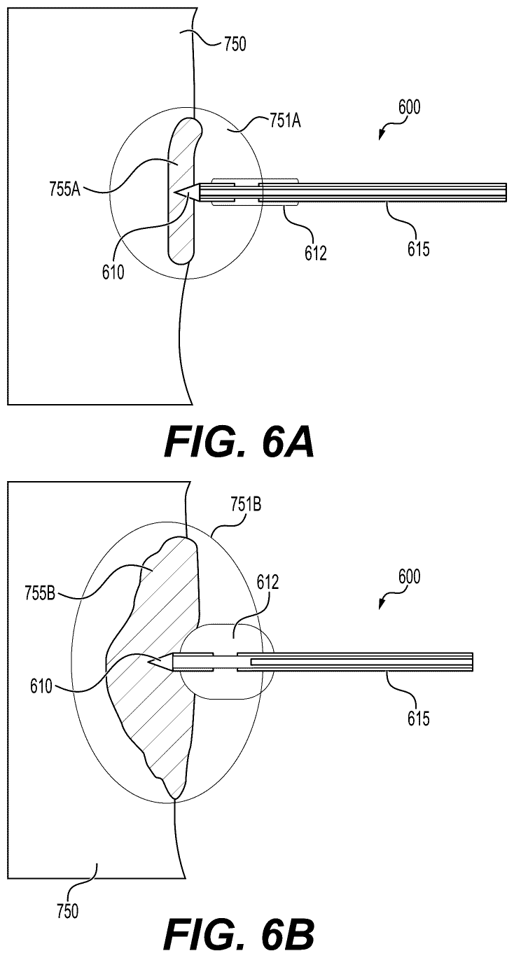

An exemplary method for treating tissue 750 according to the present disclosure is illustrated in FIGS. 6A and 6B, referring to the distal end portion of another exemplary medical device 600, which may include any of the features of medical device 100, 200, 400, and/or 500. For example, medical device 600 includes a shaft 515, a needle 610 and a balloon 612 in communication with a lumen of the shaft 615 via one or more ports. The method may include introducing the medical device 600 into a bodily lumen of a subject (e.g., a patient) and advancing the distal end of the medical device such that needle 610 is proximate a wall 750 of tissue that includes a target site 755A to be treated. For example, the bodily lumen may be the urethra, and target site 755A may be tissue within the urinary system of the subject, such as tissue of the bladder or the kidney. Target site 755A may include tissue that is cancerous or suspected of being cancerous. For example, target site 755A may include a tumor. Medical device 600 may be positioned such that needle 610 at least partially is inserted into the tissue at target site 755A.

Next, a cryogenic fluid may be supplied to needle 610 through the lumen of shaft 615, via a tube as discussed above in connection to FIGS. 3A-3D. At this step, the distal end of the tube may be adjacent to needle 610, e.g., adjacent to the proximal end of needle 610 and distal to a port of medical device 600 that is in communication with balloon 612. Thus, the port is effectively closed off to the fluid by that wall of the tube, and balloon 612 may remain deflated as shown in FIG. 6A. At the same time, the cryogenic fluid may cause needle 610 to freeze or otherwise rapidly decrease in temperature, which in turn may at least partially freeze tissue at target site 755A in contact and proximate needle 610. This is illustrated in FIG. 6A as a region 751A of tissue that includes target site 755A immediately around needle 610.

In some cases, it may be desirable to treat a larger region of tissue, e.g., a larger target site 755B, including tissues deeper within the wall 750 of tissue and/or at a greater radius relative to the site at which needle 610 is inserted. For example, target site 755B may include a relatively large tumor and/or a medical professional may seek to treat margins of tissue about a tumor. To increase the region 751B of tissue being treated, medical device 600 may be further advanced to insert needle 610 further into the tissue, shown in FIG. 6B. Additionally, the tube within the lumen of shaft 615 may be moved proximally so that the distal end of the tube is proximal to the port, allowing cryogenic fluid to flow through the tube and through the port to inflate balloon 612. Expansion of balloon 612 causes balloon 612 to press against surrounding tissue and freeze tissue due to the cold temperature of balloon 612 by the cryogenic fluid within. The tissue at target site 755B and within the larger region 751B thus may be treated using the second configuration of medical device 600 by inflation of balloon 612.

Without being bound by theory, it is believed that freezing tissue kills or ablates cells, e.g., via dehydration. Cryoablation utilizes Joule-Thompson thermodynamics to cause rapid freezing. For example, a rapid drop in temperature to freezing temperatures afforded by the medical devices herein may induce ice crystal formation in tissue, e.g., into a region (generally described as an “iceball”) at and proximate a target site. Intracellular and extracellular ice formed in the core of the region may penetrate and damage cells. On the edge of the iceball region, a hyperosmotic environment may form and dehydrates cells. On subsequent rapid thawing by removal of the medical device from the tissue, a hypotonic environment may form, wherein water may rush back into the cells, rupturing the cell membranes and ablating the cells.

The medical devices herein may be used to treat multiple target sites in one treatment session. For example, the needle at the distal end of the medical device may be inserted into different target sites, e.g., sequentially, while the balloon remains deflated. Regions of tissue proximate the needle may undergo cryoablation as discussed above, e.g., to damage and kill cells that are cancerous or suspected of being cancerous. The needle may be inserted deeper into tissue to treat areas deeper within the tissue wall. For example, in the case of multiple tumors, relatively smaller tumors may be treated by insertion of the needle and supply of cryogenic fluid to the needle.

For relatively larger areas of tissue, including relatively larger tumors, the balloon of the device may be inflated to increase the region of tissue being frozen. Thus, the balloon may be inflated through the port of the shaft of the medical device as discussed above, e.g., by moving the tube supplying the cryogenic fluid proximally along the lumen of the shaft. Following the end of the treatment session, the balloon may be deflated, e.g., by terminating the supply of cryogenic fluid and by applying negative pressure to withdraw the fluid from the balloon and through the lumen of the shaft.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A medical device comprising:
a shaft defining a lumen and having a distal end portion that includes an inflatable balloon, wherein a needle closes a distal end of the shaft; and
a tube movable along the lumen, the tube being in fluid communication with the balloon and configured to supply a cryogenic fluid to the needle and to the balloon;
wherein the medical device is movable between a first configuration in which a distal end of the tube abuts a proximal end of the needle while the balloon is deflated, and a second configuration in which the distal end of the tube is proximal to the balloon, wherein supplying the cryogenic fluid through the tube inflates the balloon in the second configuration.

2. The medical device of claim 1, wherein a wall of the distal end portion of the shaft includes at least one port that provides fluid communication between the lumen and the balloon.

3. The medical device of claim 2, wherein the at least one port includes two ports disposed opposite one another.

4. The medical device of claim 1, wherein the balloon when inflated is symmetric about the shaft.

5. The medical device of claim 1, wherein the balloon when inflated is asymmetric about the shaft.

6. The medical device of claim 1, wherein the balloon comprises polyimide, polyamide, polyurethane, nylon, or polyethylene terephthalate.

7. The medical device of claim 1, wherein the balloon has a length ranging from about 8 mm to about 15 mm.

8. The medical device of claim 1, wherein the balloon has a diameter ranging from about 8 mm to about 15 mm.

9. The medical device of claim 1, wherein the balloon when inflated has an oblong shape.

10. The medical device of claim 1, wherein the medical device comprises a handle at a proximal end of the shaft, the handle including a valve configured to control a flow of the cryogenic fluid.

11. The medical device of claim 10, wherein the valve is a pressure relief valve.

12. A medical device comprising:
a shaft defining a lumen and having a distal end portion that includes a port and an inflatable balloon, wherein a needle closes a distal end of the shaft proximate the balloon and the port; and
a tube movable along the lumen, the tube being in fluid communication with the balloon and configured to supply a cryogenic fluid to the needle and to the balloon;
wherein the medical device is movable between a first configuration in which a distal end of the tube abuts the needle while the balloon is deflated, and a second configuration in which the distal end of the tube is proximal to the port, wherein supplying the cryogenic fluid through the tube inflates the balloon in the second configuration.

13. The medical device of claim 12, wherein the at least one port includes two ports.

14. The medical device of claim 12, wherein the balloon when deflated has a plurality of preformed folds, the balloon being configured to inflate with a bellows shape.

15. A method of treating a subject, the method comprising:
introducing a medical device into a bodily lumen of the subject, wherein the medical device includes a shaft defining a lumen with a distal end portion that includes an inflatable balloon, wherein a needle closes a distal end of the shaft proximate the balloon;
at least partially inserting the needle into a target tissue site;
supplying a cryogenic fluid to at least one of the needle or the balloon through a tube movable along the lumen;
wherein the cryogenic fluid freezes at least a portion of tissue in contact with the needle and/or the balloon.

16. The method of claim 15, wherein the cryogenic fluid comprises nitrogen, nitrous oxide, argon, krypton, or a hydrocarbon refrigerant.

17. The method of claim 15, wherein a distal end of the tube is adjacent to a proximal end of the needle when supplying the cryogenic fluid.

18. The method of claim 15, wherein a distal end of the tube is proximal to the balloon when supplying the cryogenic fluid, such that the balloon inflates.

19. The method of claim 18, wherein supplying the cryogenic fluid to at least one of the needle or the balloon through the tube includes:
supplying the cryogenic fluid through the tube while the balloon is deflated and a distal end of the tube abuts a proximal end of the needle, the distal end of the tube being distal to a port of the shaft in fluid communication with the balloon;
moving the tube in a proximal direction such that the distal end of the tube is proximal to the port; and
supplying the cryogenic fluid through the tube to inflate the balloon.

20. The method of claim 15, wherein the bodily lumen is a urethra, and the target tissue site is in a bladder or a kidney.
